SEQUENCE LISTING (17 sequences)

SEQ ID NO: 1 - DNA, 15 bp, Artificial Sequence
  Oligo 1 template
  misc_feature (15)..(15): Blocked at 3′ end with a C3 Spacer
ttgagcgtgaactcg

SEQ ID NO: 2 - DNA, 15 bp, Artificial Sequence
  Oligo 2 template
  misc_feature (15)..(15): Blocked at 3′ end with a C3 Spacer
atgagcgtgaactcg

SEQ ID NO: 3 - DNA, 15 bp, Artificial Sequence
  Oligo 3 template
  misc_feature (15)..(15): Blocked at 3′ end with a C3 Spacer
ctgagcgtgaactcg

SEQ ID NO: 4 - DNA, 15 bp, Artificial Sequence
  Oligo 4 template
  misc_feature (15)..(15): Blocked at 3′ end with a C3 Spacer
gtgagcgtgaactcg

SEQ ID NO: 5 - DNA, 20 bp, Artificial Sequence
  Oligo 5 template
atcacgcgagttcacgctca

SEQ ID NO: 6 - DNA, 21 bp, Artificial Sequence
  Oligo 6 template
atcacgcgagttcacgctcac

SEQ ID NO: 7 - DNA, 14 bp, Artificial Sequence
  Oligo 7 template
  misc_feature (11)..(14): n is a, c, g, or t
  misc_feature (14)..(14): Blocked at 3′ end with a C3 Spacer
gtgagcgtgannnn

SEQ ID NO: 8 - DNA, 12 bp, Artificial Sequence
  Oligo 8 template
  misc_feature (9)..(12): n is a, c, g, or t
  misc_feature (12)..(12): Blocked at 3′ end with a C3 Spacer
gtgagcgtnnnn

SEQ ID NO: 9 - DNA, 10 bp, Artificial Sequence
  Oligo 9 template
  misc_feature (7)..(10): n is a, c, g, or t
  misc_feature (10)..(10): Blocked at 3′ end with a C3 Spacer
gtgagcnnnn

SEQ ID NO: 10 - DNA, 20 bp, Artificial Sequence
  Oligo 10 template
  misc_feature (1)..(1): Biotin modification on 5′ end
atcacgcgagttcacgctca

SEQ ID NO: 11 - DNA, 21 bp, Artificial Sequence
  Oligo 11 template
  misc_feature (1)..(1): Biotin modification on 5′ end
atcacgcgagttcacgctcac

SEQ ID NO: 12 - DNA, 22 bp, Artificial Sequence
  Oligo 12 template
  misc_feature (1)..(1): Biotin modification on 5′ end
atcacgcgagttcacgctcacc

SEQ ID NO: 13 - DNA, 15 bp, Artificial Sequence
  Oligo 13 template
  misc_feature (15)..(15): Blocked at 3′ end with a C3 Spacer
ggtgagcgtgaactc

SEQ ID NO: 14 - DNA, 11 bp, Artificial Sequence
  template strand
agtccagtagt

SEQ ID NO: 15 - DNA, 21 bp, Artificial Sequence
  growing polynucleotide using a template strand
ctactggactgtgcaaacaga

SEQ ID NO: 16 - DNA, 11 bp, Artificial Sequence
  template strand
tttgcactgtc

SEQ ID NO: 17 - DNA, 11 bp, Artificial Sequence
  template strand
ctactggactg

(19) United States
(12) Patent Application Publication
(10) Pub. No.: US 2021/0261999 A1
(43) Pub. Date: Aug. 26, 2021

(54) SOLID-PHASE SYNTHESIS OF POLYNUCLEOTIDES USING A TEMPLATE ARRAY

(71) Applicants: Richard FUISZ, Washington, DC (US); Alan TOMUSIAK, Stanford, CA (US); TEMPLANUCLEICS, INC., Palo Alto, CA (US)

(72) Inventors: Richard FUISZ, Washington, DC (US); Alan TOMUSIAK, Stanford, CA (US)

(21) Appl. No.: 17/254,214

(22) PCT Filed: Jun. 20, 2019
(86) PCT No.: PCT/US2019/038222

§ 371 Date: Dec. 18, 2020

Related U.S. Application Data
(60) Provisional application No. 62/688,964, filed on Jun. 22, 2018.

(51) Int. Cl. C12P 19/34; B01L 3/00; B01L 7/00

(52) U.S. Cl. CPC C12P 19/34; B01L 2400/043; B01L 7/52; B01L 3/50851

(57) ABSTRACT

Provided herein are methods and systems for synthesizing polynucleotides using a solid support and a template array. Also provided herein are methods of selecting templates for the array.

### FIELD

The present disclosure relates generally to methods and systems for synthesizing polynucleotides, and more specifically to methods and systems for synthesizing polynucleotides attached to a solid substrate, using a template array.

### CROSS-REFERENCE TO RELATED APPLICATIONS

This application claims priority to U.S. Provisional Patent Application No. 62/688,964, filed Jun. 22, 2018.

### BACKGROUND

Current methods of synthesizing polynucleotides include chemical routes using phosphoramidite chemistry, and enzymatic ligation assembly from small oligonucleotide fragments. Phosphoramidite methods require organic solvents and many reactants over multiple steps to add a single nucleotide. The number of steps and types of reagents affect overall coupling efficiency for each cycle, which in turn limits the total size of polynucleotides that can be synthesized. Further, some of the organic solvents and reagents can be hazardous and require special handling and disposal. In enzymatic ligation methods, the intended polynucleotide sequence is broken up into overlapping fragments of ˜50 bases each. These fragments are synthesized as single stranded oligonucleotides in separate tubes or together on a microarray, the fragments combined to anneal the overlapping regions, and annealed product ligated to produce the polynucleotide. These complex steps can result in time delays and limits the type of sequences that can be synthesized, as repetitive sequences may be difficult to assemble in the correct order.

Thus, what is needed in the art are new methods of synthesizing polynucleotides.

### BRIEF SUMMARY

In one aspect, provided herein is a method of extending a primer to generate a polynucleotide. This method includes providing a primer with a 5′ fixed end bound to a solid substrate, and a 3′ growing end; providing an array that has a plurality of template wells, wherein at least some of these wells each have two or more unique templates in solution; and contacting the solid substrate with the solution of one of the wells that has two or more unique templates. In the contacted solution, one of unique templates is a complementary template that has a complementary region, and the sequence of the complementary region is complementary to the growing end of the primer; and at least one of the unique templates is a non-complementary template, which has a sequence that is not complementary to the growing end of the primer. The method also includes hybridizing the growing end of the primer and the complementary region of the complementary template to provide an overhang of the complementary template, wherein the overhang has one or more nucleotides; extending the growing end of the primer by the addition of one or more nucleotides, wherein nucleotides added are complementary to nucleotides of the overhang; and dehybridizing the growing end and the complementary region. In some variations, these steps are repeated to generate the polynucleotide.

In some variations, the one or more nucleotides are added by a template-dependent DNA polymerase. In some variations, the method includes heating the solution in the hybridization step to hybridize the growing end and the complementary region. In certain variations, the solution is heated to within 20° C. of the melting temperature (Tₘ) of the complementary template.

In another aspect, provided herein is a system for extending a growing primer to generate a polynucleotide. This system includes a growing primer with a 5′ fixed end bound to a solid substrate, and a 3′ growing end; and a template array that has a plurality of template wells, wherein at least some of these wells each have two or more unique templates in solution. The solution of at least one of the template wells of this array comprises two or more unique templates, wherein one of the unique templates is a complementary template that has a complementary region, and the sequence of the complementary region is complementary to the growing end of the primer; and at least one of the unique templates is a non-complementary template with a sequence that is not complementary to the growing end of the primer. This system also includes a positioning apparatus that is configured to contact the solid substrate with the solution of a well.

In some variations, the system comprises no more than one million template wells each comprising two or more unique templates in solution.

In some variations, the template wells are incorporated into a removable cartridge.

In some variations, the polynucleotide is at least 300 bases in size. In certain variations, the polynucleotide comprises a GC content of 80% or greater, wherein GC content is the percentage of the total bases in the polynucleotide that comprise either guanine or cytosine. In some variations, the polynucleotide comprises fewer than 6 deletion errors. In certain variations, the polypeptide is produced with an accuracy rate of 98% or greater.

In some variations, the two or more unique templates independently comprise between 6 bases to 12 bases. In certain variations, the one or more non-complementary templates independently have a binding energy that differs by at least 5% compared to the binding energy of the complementary template.

### DESCRIPTION OF THE FIGURES

The present application can be understood by reference to the following description taken in conjunction with the accompanying figures.

FIG. 1 is a diagram of one embodiment of a system for synthesizing polynucleotides as described herein.

FIG. 2 is a diagram of one embodiment of a method for synthesizing a polynucleotide attached to a solid support as described herein.

FIG. 3 is an image of a gel demonstrating the successful addition of four bases to a growing polynucleotide, as described in Example 1.

FIG. 4 is an image of a gel demonstrating the addition of a base to a growing polynucleotide using a 10-base template strand (10-mer), a 12-base template strand (10-mer), and a 14-base template strand (10-mer) in the presence of a mixture of other templates, as described in Example 2.

FIG. 5 is a diagram of interactions by template strands in certain embodiments, including the desired and undesired interactions with the growing primer strand and undesired interactions with other templates.

FIG. 6 is an image of a gel demonstrating two iterations of adding a single nucleotide each to the same growing primer, as described in Example 3.

### DETAILED DESCRIPTION

The following description sets forth numerous exemplary configurations, methods, parameters, and the like. It should be recognized, however, that such description is not intended as a limitation on the scope of the present disclosure.

Provided herein are methods and systems for generating a polynucleotide by extending a primer, wherein the primer has a 5′ fixed end attached to a solid substrate, and a 3′ growing end. The 3′ growing end may be extended step-wise in cycles, each cycle including hybridization of the 3′ end with a complementary template to produce an overhang region; addition of one or more free nucleotides to the 3′ growing end, wherein the added one or more nucleotides are complementary to the overhang region; and dehybridization of the newly-extended primer. These steps may be repeated in the next cycle with a template complementary to the newly-extended 3′ end, again producing an overhang region and incorporating one or more free nucleotides.

The array of templates used in these methods and systems may, in some embodiments, be designed to accommodate the synthesis of a polynucleotide of any sequence. Thus, the array may, in some embodiments, comprise a plurality of templates, wherein a portion of the templates are non-complementary to the primer, and a portion of the templates are complementary to the primer. In some embodiments, two or more of the complementary templates have a different 5′ sequence capable of generating a different overhang region upon hybridization with the primer, and therefore may be used to incorporate a different nucleotide into the growing primer. In some embodiments, as the 3′ growing end of the primer is extended the 3′ sequence changes, and one or more non-complementary templates become complementary to this new sequence. Through the methods and systems provided herein, different sequences of polypeptide may be synthesized using the same template array in some embodiments. Furthermore, in some embodiments the templates may be reused. In some embodiments, the same template is used in multiple cycles in the production of one polynucleotide, for example if a polynucleotide has a repeated sequence section. In some embodiments, the templates are reused in the production of one or more polynucleotides. For example, in some embodiments a polynucleotide is produced using a plurality of templates, and one or more of those templates is then used in the production of a different polynucleotide. In some embodiments, one or more other reagents that may be present in the solution of a well may also be reused. For example, one or more polymerases, if present, may be reused in the production of one or more polynucleotides.

In some embodiments, the array comprises a plurality of template wells, wherein at least a portion of the wells each comprise two or more unique template strands. By combining templates in the same well, the same number of templates may be maintained while reducing the overall number of wells needed. Also described herein are methods of selecting templates to be combined in one well, for example methods of selecting templates to reduce undesired interactions between two or more templates, and/or to reduce undesired interactions between templates and the growing primer. The methods and systems provided herein may in some embodiments be used to synthesize a polynucleotide faster, more efficiently, with greater fidelity, and/or of a greater size than other methods or systems, for example phosphoramidite chemistry methods, enzymatic ligation methods, or other methods.

I. Generating a Polynucleotide

In one aspect, provided herein is a system for extending the 3′ end of a primer to generate a polynucleotide, wherein the 5′ end of the primer is attached to a solid substrate. The system comprises a template array comprising a plurality of template wells, wherein at least a portion of the template wells each comprise two or more unique templates in solution.

In at least one of the template wells that comprises two or more unique templates, one of these unique templates is a complementary template, comprising a complementary region. This complementary region has a sequence that is complementary to the growing 3′ end of the primer, and therefore may, in some embodiments, hybridize with the primer. In some embodiments, hybridization of the primer and this complementary template produces an overhang of the complementary template, which may be used to add nucleotides to the 3′ end of the primer. In the same well as the complementary template, at least one of the other unique templates is a non-complementary template, with a sequence that is not complementary to the growing end of the primer. Thus, in some embodiments the array comprises at least one well that comprises one complementary template and one or more non-complementary templates in solution.

In some embodiments, the system further comprises a positioning apparatus configured to contact the solid substrate with the solution of a well. For example, in some embodiments the solid substrate may be positioned to contact the solution of a well that comprises one complementary and one or more non-complementary templates. In some embodiments, the array may be positioned to contact the solution of a well with the solid substrate, wherein the well comprises one complementary and one or more non-complementary templates. In certain embodiments, the template comprises at least one well comprising one complementary template and one non-complementary template, and the solid substrate contacts the solution of a different well (for example, a wash well or a well comprising only one template in solution).

Thus, in one aspect, provided herein is a system for extending a growing primer to generate a polynucleotide, comprising:

a growing primer comprising a 5′ fixed end and a 3′ growing end, wherein the fixed end is bound to a solid substrate;

a template array comprising a plurality of template wells, wherein at least a portion of the template wells each comprise two or more unique templates in solution,
wherein in one of the template wells, one of the two or more unique templates is a complementary template comprising a complementary region, wherein the sequence of the complementary region is complementary to the growing end; and
at least one of the two or more unique templates is a non-complementary template, wherein the sequence of the non-complementary template is not complementary to the growing end; and

a positioning apparatus configured to contact the solid substrate with the solution of a well.

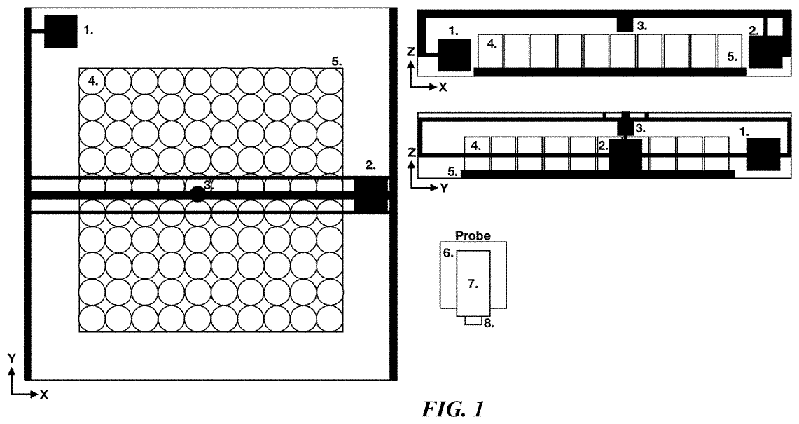

One exemplary system according to some embodiments described herein is provided in the diagram of FIG. 1. In this diagram, a positioning apparatus is configured to move a probe (3) in the X and/or Y directions as controlled by two motors (1 and 2). The probe comprises a heating element (7) and the solid support (8) bound to the primer. The template array also comprise a heating element (5) in this exemplary system. Using the two motors, the probe comprising the solid support can be positioned to contact the solution of a template well (4).

Also provided herein is a method for extending the 3′ end of a primer to generate a polynucleotide, wherein the 5′ end of the primer is attached to a solid substrate. The method includes contacting the solid substrate with the solution of a template well. In some embodiments, the contacted solution comprises a complementary template, comprising a complementary region. In certain embodiments, the complementary region hybridizes with the 3′ end of the primer, and this hybridization produces an overhang of the complementary template. In certain embodiments, the primer is extended enzymatically at the 3′ end by the addition of one or more nucleotides which are complementary to this overhang. In some embodiments, the growing end and the complementary template are then dehybridized. The steps of the method may be, in some embodiments, repeated one or more times to extend the 3′ end of the primer and produce the polynucleotide. In certain embodiments, the method comprises one or more additional steps, such as a step to wash the solid substrate, or a step comprising a chemical reaction with the growing primer (for example, photo deprotection), or a step comprising contacting the solution of a well that does not comprise a complementary template.

In some embodiments of the method provided herein, the generated polynucleotide is then combined with a reverse primer, and a complementary polynucleotide is generated. Thus, in some embodiments, after extension of the primer to generate a single stranded polynucleotide, a reverse primer is then used to generate a double stranded polynucleotide. The reverse primer, in some embodiments, has a 5′ end that is complementary to the 3′ end of the generated polynucleotide, and a 3′ growing end that is extended to generate the complementary polynucleotide. In some embodiments, the 3′ growing end of the reverse primer is extended in the presence of a DNA polymerase and one or more nucleotides.

In some embodiments of the method, after extending the primer to generate polynucleotide, the polynucleotide is cleaved from the solid support. In some embodiments, the polynucleotide is cleaved enzymatically. In certain embodiments, the polynucleotide is cleaved by a restriction enzyme, for example, by a Type IIS restriction enzyme. In certain embodiments the generated polynucleotide remains attached to the solid support, and is used in one or more subsequent reactions.

In some embodiments of the method, the template well comprising the contacted solution is one of plurality of template wells in an array, and at least a portion of the template wells of the array each comprise two or more unique templates in solution. In some embodiments, the contacted solution comprises two or more unique templates. In certain embodiments, the contacted solution comprises one complementary template, and one or more non-complementary templates.

Thus, in one aspect, provided herein is a method of extending a primer to generate a polynucleotide, comprising:
(a) providing a primer, wherein the primer has a 5′ fixed end and a 3′ growing end, wherein the fixed end is bound to a solid substrate;
(b) providing an array comprising a plurality of template wells, wherein at least a portion of the wells each comprise two or more unique templates in solution;
(c) contacting the solid substrate with the solution of a well comprising two or more unique templates,
   wherein one of the two or more unique templates is a complementary template comprising a complementary region, wherein the sequence of the complementary region is complementary to the growing end; and
   at least one of the two or more unique templates is a non-complementary template, wherein the sequence of the non-complementary template is not complementary to the growing end;
(d) hybridizing the growing end and the complementary region to provide an overhang of the complementary template comprising one or more nucleotides;
(e) extending the growing end of the primer by the addition of one or more nucleotides, wherein the one or more added nucleotides are complementary to the one or more nucleotides of the overhang region;
(f) dehybridizing the growing end and the complementary region; and
repeating steps (c) through (f) to generate the polynucleotide.

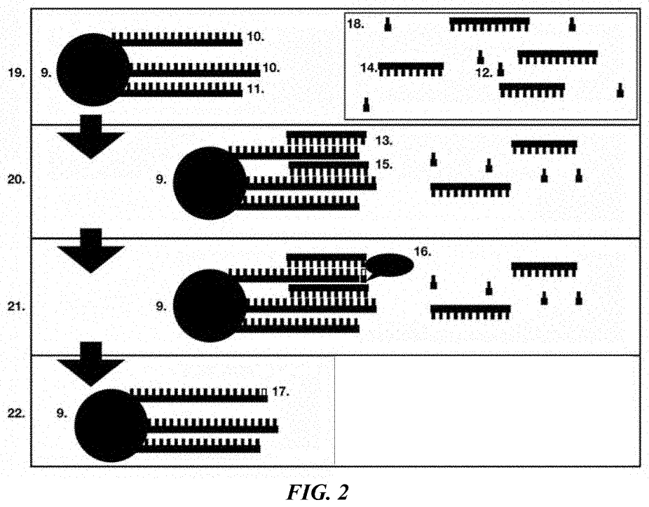

Provided in FIG. 2 is a diagram of an exemplary embodiment of the method described herein. As shown in this example, prior to contact (19), a primer with a 3′ growing end (10) is attached to a solid support (9) at the 5′ end, along with inactive primer (11). The template well (18) comprises two unique templates: a complementary template (13, unlabeled template comprising 11 bases) with a region complementary to the 3′ end of the primer, and a non-complementary template (14, comprising 9 bases). The template well also comprises a nucleotide free in solution (12). In step 20, the solid support is contacting the solution of the template well and the complementary template is hybridized to the 3′ end of the primer to produce a one-nucleotide overhang of the template (13). In the extension step (21), a polymerase extends the growing end by the addition of the nucleotide free in solution, wherein the nucleotide is complementary to the overhang. Following extension, the primer and template dehybridize to produce an extended primer (22). As the primer is extended, the 3′ sequence changes due to the addition of the nucleotide. To extend the primer again, in some embodiments the solid substrate contacts a second template well that comprises a new template complementary to this new 3′ sequence, repeating the hybridization and extension steps to generate an overhang and incorporate a second nucleotide (which may be the same or different from the first nucleotide, and is determined by the overhang region). By repeating this cycle of (a) contact with a well containing a template complementary to the newly-extended 3′ end, (b) hybridization, and (c) incorporation of a free nucleotide, the primer is extended step-wise to generate the polynucleotide. In some embodiments, the methods provided herein may be used with an array comprising a plurality of template wells, wherein at least a portion of the template wells comprise two or more unique templates.

In some embodiments, using the system or method provided herein, one nucleotide can be added to the growing 3′ end of the primer in less than 5 min, less than 4 minutes, less than 3 minutes, less than 2 minutes, less than 1 minute, less than 30 seconds, or less than 15 seconds. In certain embodiments, one cycle of contacting the solid substrate with the solution of a well comprising two or more unique templates; hybridizing the growing end of the primer and the complementary region of the template; extending the growing end of the primer by the addition of one or more nucleotides; and dehybridizing the growing end and the complementary region can be completed in less than in less than 5 min, less than 4 minutes, less than 3 minutes, less than 2 minutes, less than 1 minute, less than 30 seconds, or less than 15 seconds. In certain embodiments, this cycle can be completed in less than 1 minutes. In some embodiments, wherein a cycle of contacting, hybridizing, extending, and dehybridizing is repeated two or more times, at least 80% of the cycles, at least 85% of the cycles, at least 90% of the cycles, at least 95% of the cycles, or at least 99% of the cycles are completed in less than 5 min, less than 4 minutes, less than 3 minutes, less than 2 minutes, less than 1 minute, less than 30 seconds, or less than 15 seconds. In some embodiments, at least 80% of the cycles, at least 85% of the cycles, at least 90% of the cycles, at least 95% of the cycles, or at least 99% of the cycles are completed in less than 1 minute. In certain embodiments, method comprises one or more additional steps in one or more cycles (such as washing, or modifying the primer or polynucleotide), which may take additional time. In certain embodiments, the one or more additional steps do not take additional time.

a. Primer

The primer extended using the method and system provided herein comprises a 3′ growing end, and a 5′ fixed end bound to a solid substrate. Any suitable solid substrate may be used. For example, in some embodiments the solid substrate comprises a polymer, such as polystyrene. In some embodiments, the solid substrate comprises glass, such as controlled pore glass. In some embodiments, the solid substrate comprises a magnet, such as a magnetic bead. In certain embodiments, the solid substrate is supramagnetic. In some embodiments, the solid substrate comprises silicon. In certain embodiments, the solid substrate comprises, for example, a polymer-coated magnetic bead. The 5′ terminal nucleotide of the primer may be bound directly to the solid substrate, or may be bound through a linker group. In some embodiments, the 5′ fixed end of the primer is bound to the solid substrate through a protein binding complex, such as avidin-biotin or streptavidin-biotin. In some embodiments, the 5′ fixed end of the primer is bound to biotin, streptavidin is bound to the solid support, and the biotin and streptavidin bind to each other. Modified forms of avidin may also be used in some embodiments. In other embodiments, the 5′ fixed end of the primer is bound to the solid substrate through a chemical linker.

In some embodiments, prior to the addition of any nucleotides according to the steps of the methods provided herein, the primer comprises as few as 6 bases. In some embodiments, the primer comprises less than 6 bases prior to the addition of any nucleotides. In other embodiments, before the addition of nucleotides according to the steps of the methods provided herein, the primer comprises between 6 and 100 bases, between 6 and 50 bases, between 6 and 40 bases, between 6 and 30 bases, between 6 and 20 bases, between 6 and 15 bases, or between 6 and 10 bases. In some embodiments, as nucleotides are added to the growing end of the primer, the size of the primer increases up to the size of the polynucleotide being generated. For example, in some embodiments, the primer comprises between 6 bases to 30 kb, from 6 bases to 20 kb, from 6 bases to 10 kb, from 6 bases to 1 kb, from 6 bases to 500 bases, or from 6 bases to 100 bases.

The size of a polynucleotide (such as the polynucleotide being generated, a primer, or a template molecule) may be described herein by referring to the number of “bases” present in the covalently attached nucleotide monomer units that form said molecule. For example, a polynucleotide comprising 1000 nucleotide monomer units may be described as 1000 bases in size, or as a 1 kilobase (kb) polynucleotide. The size of a polynucleotide molecule may also be described with “-mer”. For example a template comprising 10 nucleotide monomer units may be described as a 10-mer.

The primer may be synthesized through known methods, or may be purchased from a commercial source. It should be understood that the solid support may be bound to a plurality of primer molecules of the same sequence, for example to generate a plurality of polynucleotide molecules of the same sequence at the same time.

b. Template Array

The template array of the system and method described herein comprises a plurality of template wells, wherein at least a portion of the template wells each comprise two or more unique templates in solution.

The array may comprise, for example, up to 1.1 million template wells, up to 1 million wells, up to 750 thousand template wells, up to 500 thousand template wells, up to 250 thousand template wells, up to 100 thousand template wells, up to 75 thousand template wells, up to 50 thousand template wells, up to 25 thousand template wells, up to 10 thousand template wells, up to 7,500 template wells, up to 2,500 template wells, or up to 1,000 template wells. At least a portion of the template wells each comprise two or more unique templates in solution.

The size of the template array may in some embodiments depend on the size and complexity of the templates required for the multiple cycles to produce the desired polynucleotide. For example, an array including all possible template sequences of 8 bases (using the four standard bases), where there is no template degeneracy, would require 65,536 different templates (4⁸). An array including all possible template sequences of 6 bases (using the four standard bases), with no degeneracy, would require 4,096 templates. Keeping each template separate would then require a corresponding number of wells in the array. However, by selecting certain templates to be combined in certain wells, a smaller array may be constructed containing the same total number of templates. Introducing one or more sites of degeneracy in two or more templates may also reduce the number of wells required. Furthermore, in some embodiments the templates are not uniform in size. An array comprising a mixture of relative smaller and relatively large templates requires fewer total templates to cover all possible sequences than an array comprising templates all of the relatively largest size. In addition, using a mixture of different template sizes may, in some embodiments, allow a plurality of templates to be selected that all have a Tₘ in a particular range; or for multiple templates with a Tₘ in a particular range to be combined in a single well; or for one or more templates with a Tₘ in a first range to be selected for one well, and one or more templates with a Tₘ in a second range to be selected for a different well. In some embodiments, an array comprises fewer than all possible template sequences.

Templates

In some embodiments, the sequences of two or more unique templates in the same well independently differ by at least one base. In other embodiments, the sequences of two or more unique templates in the same well independently differ by at least 2 bases, at least 3 bases, at least 4 bases, at least 5 bases, at least 6 base, at least 7 bases, at 8 eight bases, at 9 nine bases, at least 10 bases, at least 11 bases, at least 12 bases, at least 13 bases, at least 14 bases, at least 15 bases, at least 16 bases, at least 17 bases, at least 18 bases, at least 19 bases, at least 20 bases, at least 21 bases, or at least 22 bases. In some embodiments, the sequences of two or more unique templates in the same well independently have less than 90% sequence similarity, less than 85% sequence similarity, less than 80% A sequence similarity, less than 75% sequence similarity, less than 70% sequence similarity, less than 65% sequence similarity, less than 60% sequence similarity, less than 55% sequence similarity, less than 50% sequence similarity, less than 45% sequence similarity, less than 40% sequence similarity, less than 35% sequence similarity, less than 30% sequence similarity, less than 25% sequence similarity, less than 20% sequence similarity, less than 15% sequence similarity, less than 10% sequence similarity, or less than 5% sequence similarity.

In some embodiments, at least one template well comprises a complementary template. In certain embodiments, in at least one of the template wells comprising two or more unique templates, one of the unique templates is a complementary template. Each complementary template comprises two segments: a complementary region which is complementary to the 3′ end of the primer, and an overhang region. In some embodiments, the complementary region comprises at least 4 bases, at least 5 bases, at least 6 bases, at least 7 bases, at least 8 bases, at least 9 bases, at least 10 bases, at least 11 bases, at least 12 bases, at least 13 bases, at least 14 bases, at least 15 bases, at least 16 bases, at least 17 bases, at least 18 bases, at least 19 bases, or at least 20 bases complementary to the 3′ growing end of the primer. In some embodiments, the complementary region is complementary with at least a portion of the 30 bases at the 3′ end, at least a portion of the 25 bases at the 3′ end, at least a portion of the 20 bases at the 3′ end, at least a portion of the 15 bases at the 3′ end, at least a portion of the 10 bases at the 3′ end, or at least a portion of the 5 bases at the 3′ end of the primer. The overhang region is at the 5′ terminus of the complementary template. In some embodiments, the overhang region comprises between 1 to 3 nucleotides. In certain embodiments, the overhang region comprises 1 nucleotide. Upon hybridization of the complementary template and the primer, the complementary region base-pairs with the 3′ end of the primer, leaving the 5′ overhang region unpaired.

In some embodiments, at least one template well comprises a non-complementary template. In certain embodiments, wherein one of the template wells comprising two or more unique templates and one of the unique templates is a complementary template, one or more of the remaining unique templates are non-complementary. In certain embodiments, each of the remaining unique templates is non-complementary. The sequence of a complementary template has greater similarity than a non-complementary template in the same well with the 3′ growing end of the primer. Thus, for example, if the complementary template has at least 5 bases complementary to the 3′ growing end of the primer, each non-complementary template in the same well independently has no more than 4 bases complementary to the 3′ growing end of the primer. In some embodiments, each non-complementary template independently has no more than 2 bases, no more than 3 bases, no more than 4 bases, no more than 5 bases, no more than 6 bases, no more than 7 bases, no more than 8 bases, no more than 9 bases, no more than 10 bases, no more than 11 bases, no more than 12 bases, no more than 13 bases, no more than 14 bases, no more than 15 bases, no more than 16 bases, no more than 17 bases, no more than 18 bases, or no more than 19 bases complementary to the 3′ end of the primer. In some embodiments, the non-complementary template is not complementary with at least a portion of the 30 bases at the 3′ end, at least a portion of the 25 bases at the 3′ end, at least a portion of the 20 bases at the 3′ end, at least a portion of the 15 bases at the 3′ end, at least a portion of the 10 bases at the 3′ end, or at least a portion of the 5 bases at the 3′ end of the primer. In some embodiments, each non-complementary template independently has less than 90% sequence similarity, less than 85% sequence similarity, less than 80% sequence similarity, less than 75% sequence similarity, less than 70% sequence similarity, less than 65% sequence similarity, less than 60% sequence similarity, less than 55% sequence similarity, less than 50% sequence similarity, less than 45% sequence similarity, less than 40% sequence similarity, less than 35% sequence similarity, less than 30% sequence similarity, less than 25% sequence similarity, less than 20% sequence similarity, less than 15% sequence similarity, less than 10% sequence similarity, or less than 5% sequence similarity with the complementary region of the complementary template.

In some embodiments one or more of the template wells comprise one template, wherein the template is independently a complementary or non-complementary template. In some embodiments, the method further comprises contacting the solid substrate with the solution of a well comprising one template.

Each template comprises a polynucleotide, and may, for example, independently comprise from 6 bases to 21 bases, from 6 bases to 18 bases, from 6 bases to 16 bases, from 6 bases to 14 bases, from 6 bases to 12 bases, or from 6 bases to 10 bases. In some embodiments, each template independently comprises 6 bases, 7, bases, 8 bases, 9 bases, 10 bases, 11 bases, 12 bases, 13 bases, or 14 bases. In some embodiments, at least two unique templates in the array comprise a different number of bases. In certain embodiments, at least 10%, at least 20%, at least 30%/6, or at least 40%/o of the unique templates of the array comprise a different number of bases, compared to the remaining unique templates. In certain embodiments, the two or more unique templates of a well independently comprise between 6 bases to 14 bases, between 6 bases to 12 bases, between 6 bases to 10 bases, between 8 bases to 14 bases, or between 8 bases to 12 bases.

In certain embodiments, one or more templates comprise a blocking group at the 3′ end, which prevents extension of the template in the presence of a polymerase. In certain embodiments, each template comprises a blocking group at the 3′ end, which prevents extension of the template in the presence of a polymerase. Any suitable blocking group may be used. In some embodiments, the 3′ blocking group is a C3 Spacer. In other embodiments, the 3′ blocking group is phosphorylation.

Each template may independently comprise standard bases, non-standard bases, or any mixtures thereof. Standard bases may include, for example, adenine, cytosine, guanine, and thymine. Templates comprising non-standard bases may comprise, for example, 2-aminopurine; 2,6-diaminopurine; 5-bromo-deoxyuridine; deoxyuridine; inverted deoxythymidine; inverted dideoxythymidine; dideoxycytidine; 5-methyl deoxycytidine; deoxyinosine; 5-hydroxybutynl-2′-deoxyuridine; 8-aza-7-deazaguanosine; 5-nitroindole; isodeoxycytidine; isodeoxyguinidine; fluorinated adenine; fluorinated cytosine; fluorinated guanine; or fluorinated thymine, or any combinations thereof.

In some embodiments, each template independently has a melting temperature (Tₘ) that is within 1° C. of the temperature of the solution during hybridization. In some embodiments, each template independently has a Tₘ that is within 2° C., within 3° C., within 4° C., within 5° C., within 10° C., within 15° C., within 20° C., within 1° C. to 20° C., within 1° C. to 15° C., within 1° C. to 10° C., or within 1° C. to 5° C. of the temperature of the solution during hybridization. In certain embodiments, each template independently has a Tₘ that is from 20° C. below to 10° C. above the temperature of the solution during hybridization, or from 20° C. below to 5° C. above the temperature of the solution during hybridization, or from 5° C. below to 20° C. above the temperature of the solution during hybridization, or from 2° C. below to 10° C. above the temperature of the solution during hybridization. In some embodiments, the temperature of the solution during hybridization is 20° C. or more below the Tₘ of the complementary template. In certain embodiments, the temperature of the solution during hybridization is no more than 20° C. below the Tₘ of the complementary template. In other embodiments, the temperature of the solution during hybridization is no more than 15° C. below the Tₘ of the complementary template. In still further embodiments, the temperature of the solution during hybridization is no more than 10° C. below the Tₘ of the complementary template. In certain embodiments, the temperature of the solution in the well is thermocycled for repeated melting and re-hybridization of a template and primer. Thus, for example, in some embodiments, the method further comprises repeated heating and cooling of the solution during to the hybridization step to hybridize the growing end and the complementary region. In certain embodiments, using a complementary template with a Tₘ within 1° C. to 10° C. of the solution during hybridization reduces undesired binding from interfering with hybridization of the complementary region and primer. In certain embodiments, using a complementary template with a Tₘ within 1° C. to 10° C. of the solution during hybridization improves fidelity when synthesizing repeat regions in a polynucleotide.

In some embodiments, each template independently has a melting temperature (Tₘ) that is within 1° C. of the temperature of the solution during extension of the 3′ end of the primer. In some embodiments, each template independently has a Tₘ that is within 2° C., within 3° C., within 4° C., within 5° C., within 10° C., within 15° C., within 20° C., within 1° C. to 20° C., within 1° C. to 15° C., within 1° C. to 10° C., or within 1° C. to 5° C. of the temperature of the solution during extension. In certain embodiments, each template independently has a Tₘ that is from 20° C. below to 10° C. above the temperature of the solution during extension, or from 20° C. below to 5° C. above the temperature of the solution during extension, or from 5° C. below to 20° C. above the temperature of the solution during extension, or from 2° C. below to 10° C. above the temperature of the solution during extension. In some embodiments, the temperature of the solution during extension is 20′C or more below the Tₘ of the complementary template. In certain embodiments, the temperature of the solution during extension is no more than 20° C. below the Tₘ of the complementary template. In other embodiments, the temperature of the solution during extension is no more than 15° C. below the Tₘ of the complementary template. In still further embodiments, the temperature of the solution during extension is no more than 10° C. below the Tₘ of the complementary template. In certain embodiments, using a complementary template with a Tₘ within 1° C. to 10° C. of the solution during extension improves fidelity when synthesizing repeat regions in a polynucleotide.

In certain embodiments, the temperature during extension is the same as the temperature during hybridization.

In some embodiments, in a well comprising two or more templates, each template has the same Tₘ. In certain embodiments, in a well comprising two or more templates, each template has a Tₘ within the same 2° C. range, or within the same 3° C. range, or within the same 4° C. range. In certain embodiments, the templates in one or more wells of the array have a Tₘ within a certain range (e.g., a 2° C. range, a 3° C. range, or a 4° C. range), and the templates in one or more other wells of the array have a Tₘ in a different range. Thus, for example, in some embodiments, the templates in one or more wells have a Tₘ within the same 2° C. range, or within the same 3° C. range, or within the same 4° C. range; and the templates in one or more other wells have a Tₘ that differs by at least 1° C., at least 2° C., at least 3° C., at least 4° C., at least 5° C., at least 6° C., at least 7° C., or at least 8° C. from the range.

In some embodiments, the Tₘ is calculated according to the following formula:

(4° C.)*(#G and C nucleotides)+(2° C.)*(#A and T nucleotides)=Tₘ

Other Tₘ equations may be used in certain embodiments. For example, in certain embodiments, the Tₘ is calculated using a nearest neighbors approach, which may take into account, for example, the calculated interactions between bases and their adjacent nucleotides. In some embodiments, the Tₘ may be affected by the conditions of an aqueous buffer, for example pH, salt concentration, salt identity, and template concentration. For example, the nearest neighbors approach to calculating Tₘ may take into account pH, salt concentration, salt identity, and template concentration.

Template Wells

The solution of each template well is independently aqueous buffer. In some embodiments, the aqueous buffer is a buffer solution compatible with a polymerase, for example a template dependent DNA polymerase. Any buffer suitable for extension by a polymerase may be used. For example, in some embodiments the buffer comprises 50 mM NaCL, 10 mM Tris-HCl, 10 mM MgCl₂, and 1 mM DTT at pH 7.9. The buffer may be prepared according to methods know in the art, or may be obtained from a commercial source. In some embodiments, the solution further comprises one or more additional components, such as one or more additional compounds and/or one or more enzymes. In some embodiments, the solution comprises proline. In some embodiments, including proline in the buffer may help stabilize the polymerase, extending the time period or number of cycles that the enzyme is active. In some embodiments, the solution comprises phosphatase. In certain embodiments, phosphatase may degrade pyrophosphate produced as a byproduct of incorporating nucleotides into the growing primer, and may increase the number of reuse cycles of the solution. In some embodiments, one or more wells independently comprise a hydrophobic liquid, which may be used, for example, to decrease evaporation of the aqueous buffer. For example, in some embodiments, one or more wells independently comprise mineral oil.

In some embodiments, the solution of a well further comprises one or more nucleotides, wherein the nucleotides are free in the solution. For example, in some embodiments, at least a portion of wells comprising one or more templates further comprise one or more nucleotides, wherein the nucleotides are free in solution. In some embodiments, the solution of the well contacting the solid substrate comprises one or more nucleotides free in solution. The one or more nucleotides may comprise standard bases, or may comprise non-standard bases, as described above. In some embodiments, the base of the nucleotide is modified with a ligand, for example a linker attached to a binding group such as biotin. In some embodiments, the 3′ hydroxyl of the nucleotide is modified, such as by an ester or ether. In some embodiments the solution comprises a nucleotide with a reversible terminator, wherein the 3′-OH is modified with a cleavable moiety. The moiety may be chemically or photo cleavable. The inclusion of a reversible terminator nucleotide may be useful, for example, in generating a polynucleotide with a series of repeating bases in the sequence. The use of reversible terminator nucleotides may, in some embodiments, prevent unintended and/or uncontrolled multiple additions of the same nucleotide from being added in one cycle. Instead, in some embodiments, the reversible terminator moiety allows for only one addition of the nucleotide to the 3′ end of the primer, after which the moiety must be removed before another nucleotide can be added.

In some embodiments, the solution of one or more wells each independently comprise one nucleotide free in solution. For example, in some embodiments, the solution of the well contacting the solid support comprises one nucleotide free in solution. In some embodiments, the one nucleotide is complementary to the 5′ terminal nucleotide of the complementary template. In some embodiments, all of the templates present in the solution of one well have the same 5′ terminal nucleotide, the solution further comprises one free nucleotide, and the free nucleotide is complementary to the 5′ terminal nucleotide of the templates.

In some embodiments, the solution of one or more wells independently comprises a polymerase, such as a template dependent DNA polymerase. In certain embodiments, the solution of the well contacting the solid substrate comprises a polymerase. A variety of different template dependent DNA polymerases may be used in the method and system described herein. The polymerase may be a naturally occurring polymerase, or may be a non-natural or engineered polymerase. In some embodiments, the polymerase is free in solution, while in other embodiments the polymerase is bound to a solid substrate. In certain embodiments, the template dependent DNA polymerase is the Klenow Fragment of E. Coli Polymerase I (Klenow). In certain embodiments, Klenow requires a shorter segment of adjacent nucleotides to bind and incorporate a deoxyribonucleotide triphosphate (dNTP) into the growing primer than other DNA polymerases, which may allow the use of shorter template strands than other DNA polymerases. The use of shorter template strands requires a lower total number of templates to cover all possible primer sequences than if longer template strands are used. In some embodiments, the solution comprises a polymerase other than Klenow.

The array may further comprise one or more additional wells. In some embodiments, the array comprises one or more wash wells, which may be used, for example, to remove residual template or nucleotide on the solid substrate after the solid substrate no longer contacts the solution of a template well. In some embodiments, the one or more wash wells independently comprise aqueous buffer. In some embodiments, the array comprises one or more reaction wells for the modification of the primer. For example, in certain embodiments the array comprises one or more reaction wells for the removal of a reversible terminator moiety from the 3′ end of a growing primer. In other embodiments, the array comprises one or more reaction wells to free the generated polynucleotide from the solid substrate.

In some embodiments, the method further comprises washing the solid substrate. Thus, in some embodiments, the method further comprises contacting the solid substrate with the solution of a wash well to wash the solid substrate. In certain embodiments, the method comprises washing the solid substrate one or more times, for example washing the substrate after one or more steps of the method. In some embodiments, the method further comprises washing the solid substrate after dehybridizing the growing end and the complementary region. In some embodiments, the method further comprises washing the solid substrate after generating the polynucleotide. In certain embodiments, the method further comprises washing the solid substrate prior to contacting the solid substrate with the solution of a well comprising two or more templates.

In certain embodiments, the method further includes contacting the solid substrate with the solution of a reaction well to modify the primer. In certain embodiments, the method comprises modifying the primer one or more times, for example modifying the primer after one or more steps of the method. In some embodiments, the primer is modified in one or more ways, for example in two sequential reactions. In some embodiments, the solid substrate is contacted with the solution of one reaction well to modify the primer, then contacted with the solution of another reaction well to modify the primer. In some embodiments, the modification is the removal of a reversible terminator moiety. In some embodiments, the method further comprises modifying the primer after dehybridizing the growing end and the complementary region. In some embodiments, the method further comprises modifying the primer prior to contacting the solid substrate with the solution of a well comprises two or more unique templates.

In some embodiments, the method further comprises modifying the polynucleotide after it is generated. In other embodiments, the method further includes contacting the solid substrate to free the generated polynucleotide from the solid substrate. Freeing the polynucleotide may be accomplished through a chemical reaction or by enzymatic means. For example, in some embodiments, the polynucleotide is cleaved from the solid substrate using a restriction enzyme, such as a Type IIS restriction enzyme. Thus, in some embodiments, the method further comprises freeing the polynucleotide after it is generated. In some embodiments, the freed polynucleotide undergoes one or more additional reactions. In other embodiments, the polynucleotide remains attached to the solid support and undergoes one or more additional reactions.

In some embodiments, the system further comprises a heating element. In certain embodiments, the system further comprises two or more heating elements. A heating element may be attached to the solid support, or to the array. In some embodiments, the system comprises a heating element configured to control the temperature of the solution of one or more wells. For example, in some embodiments the system comprises a heating element configured to control the temperature of the solution of a plurality of template wells. In certain embodiments, the system comprises a plurality of heating elements, wherein the temperature of the solution of each template well is separately controlled by a heating element. In some embodiments, the system comprises a heating element configured to control the temperature of the solid support. In certain embodiments, the system comprises a heating element configured to control the temperature of a template well, wherein the heating element is attached to the solid support. For example, in some embodiments, the system comprises a heating element attached to the solid support, and upon contacting the solid support with the solution of a well, the heating element controls the temperature of the solution. In some embodiments, the system comprises a heating element attached to the array, wherein the heating element is configured to control the temperature of one or more wells of the array. In some embodiments, the system comprises a heating element attached to the solid support, and one or more heating elements attached to the array. For example, the diagram of FIG. 1 demonstrates an exemplary system with a heating element attached to the array (5), configured to control the temperature of one or more wells of the array; and a heating element attached to the solid support (7). In this example, the heating element on the array may, in some embodiments, maintain the temperature of a plurality of wells within a set range (for example, relatively colder than the hybridization temperature), and upon contacting the solid support with the solution of a well, the heating element attached to the solid support heats the solution of that well such that hybridization between the primer and a complementary template can occur. In some embodiments, the array comprises a plurality of heating elements attached to the array, and a heating element attached to the solid support.

In some variations, one or more template wells are incorporated into a removable cartridge. Thus, for example, in some embodiments, the array comprises a removable cartridge, wherein the removable cartridge comprises one or more template wells. In certain embodiments, a first cartridge comprising one or more template wells can be removed from the array, and a second cartridge comprising one or more template wells, which may be the same or different from the first cartridge, is placed into the array. In still further embodiments, one or more wells, which may include, for example, one or more template wells, one or more wash wells, or one or more other reaction wells, remain in the array when the first cartridge is removed.

In some embodiments, the method of extending a primer further comprises heating the solution to hybridize the growing end and the complementary region. For example, in some embodiments, the method of extending a primer further comprises heating the solution to hybridize the growing end and the complementary region. In some embodiments, the solution is heated and cooled repeatedly to hybridize the growing end and the complementary region. In certain embodiments, the solution is heated to within 25° C., to within 20° C., to within 15° C., to within 10° C., to within 5° C., or to within 1° C., of the Tₘ of the complementary template. In certain embodiments, the solution is heated to within 20° C. of the Tₘ of the complementary template.

In some embodiments, the method of extending a primer further comprises cooling the solution to dehybridize the growing end and the complementary region. For example, in some embodiments, the method of extending a primer further comprises cooling the solution to dehybridize the growing end and the complementary region. In some embodiments, the solution is cooled to less than 15° C., to less than 20° C., to less than 25° C., to less than 30° C., to less than 35° C., to less than 40° C., to less than 45° C., or to less than 50° C. below the Tₘ of the complementary template.

In some embodiments of the method and system described herein, one or more template wells that are not being contacted by the solid substrate are maintained at less than 15° C., less than 10° C., less than 5° C., less than 0° C., less than −10° C., less than −20° C., or less than −30° C.

In some embodiments, the system further comprises a positioning apparatus configured to contact the solid substrate with the solution of a well. In some embodiments, the positioning apparatus is configured to position one or more wells of the array to contact the solid substrate with the solution of a well. In some embodiments, the positioning apparatus is configured to position the solid substrate to contact the solution of a well. In certain embodiments, the positioning apparatus is configured to position both the solid substrate and one or more wells of the array, to contact the solid substrate with the solution of a well. In still further embodiments, the system comprises two or more separate positioning apparatus, which may be configured to position the solid substrate, one or more wells of the array, or both, to contact the solid substrate with the solution of a well. Any suitable positioning apparatus may be used. In some embodiments, the system comprises stationary template array and a positioning apparatus to position the solid support, for example, an apparatus similar to a 3-D printer or an XY plotter. In some embodiments, the positioning apparatus is configured to position the solid substrate to contact the solution of the well, and at least a portion of the positioning apparatus is magnetic. For example, in some embodiments, the positioning apparatus comprises a magnetic needle, the solid substrate comprises a magnetic bead, and the magnetic bead is magnetically attached to the magnetic needle. In certain embodiments, the system comprises a plurality of solid substrates. For example, in some embodiments the solid substrate is a bead with a bound primer, as described above, and a plurality of beads with bound primer are used in the system. In certain embodiments, a plurality of beads with bound primer contact the solution of a well. For example, in some embodiments the system comprises a positioning apparatus that positions a plurality of beads to contact the beads with the solution of a well.

II. Polynucleotide

In some embodiments, the polynucleotide generated using the method or system provided herein has a sequence that may be found in a eukaryotic or prokaryotic organism. In some embodiments, the sequence of the polynucleotide is a bacterial, mammalian, fungal, or plant sequence. In certain embodiments, the polynucleotide sequence is a bacterial plasmid. I In certain embodiments, at least a portion of the sequence of a polynucleotide generated using the method or system provided herein may be found in a eukaryotic or prokaryotic organism, such as a bacterium, mammal, fungus, or plant. In certain embodiments, the polynucleotide sequence is a an engineered bacterial plasmid.

In some embodiments, the single stranded polynucleotide generated by the method or system described herein is at least one kilobase in size. In certain embodiments, the polynucleotide is at least 1 kb, at least 2 kb, at least 3 kb, at least 4 kb, at least 5 kb, at least 6 kb, at least 7 kb, at least 8 kb, at least 9 kb, at least 10 kb, at least 11 kb, at least 12 kb at least 13 kb, at least 14 kb, at least 15 kb, at least 16 kb, at least 17 kb, at least 17 kb, at least 18 kb, at least 19 kb, or at least 20 kb in size. In certain embodiments, the polynucleotide is at least 1 kb, and is generated with a higher level of accuracy compared to the intended sequence using the method or system described herein would be using another method, such as phosphoramidite chemistry or enzymatic ligation. For example, in certain embodiments, a polynucleotide at least 1 kb, at least 5 kb, at least 10 kb, at least 15 kb, or at least 20 kb in size can be synthesized with a higher level of accuracy (compared to the intended sequence) than methods using phosphoramidite chemistry or enzymatic ligation.

While in some embodiments the polynucleotide may be, for example, at least one kilobase (kb) in size, in certain embodiments the method or system provided herein may be used to prepare a smaller polynucleotide. For example, in some embodiments, the polynucleotide is at least 50 bases, at least 100 bases, at least 150 bases, at least 200 bases, at least 250 bases, at least 300 bases, at least 350 bases, at least 400 bases, at least 450 bases, at least 500 bases, at least 550 bases, at least 600 bases, at least 650 bases, at least 700 bases, at least 750 bases, at least 800 bases, at least 850 bases, at least 900 bases, at least 950 bases, or at least 999 bases in size. In some embodiments, the polynucleotide is 50 bases or less, 100 bases or less, 150 bases or less, 200 bases or less, 250 bases or less, 300 bases or less, 350 bases or less, 400 bases or less, 450 bases or less, 500 bases or less, 550 bases or less, 600 bases or less, 650 bases or less, 700 bases or less, 750 bases or less, 800 bases or less, 850 bases or less, 900 bases or less, 950 bases or less, or 999 bases or less in size. In some embodiments, the polynucleotide is between 100 bases to 30 kb, between 100 bases to 25 kb, between 100 bases to 20 kb, between 100 bases to 15 kb, between 100 bases to 10 kb, between 100 bases to 5 kb, between 100 bases to 1 kb, between 1 kb to 30 kb, between 1 kb to 25 kb, between 1 kb to 20 kb, between 1 kb to 15 kb, between 1 kb to 10 kb, between 1 kb to 5 kb, between 5 kb to 30 kb, between 5 kb to 25 kb, between 5 kb to 20 kb, between 5 kb to 15 kb, between 5 kb to 10 kb, between 10 kb to 30 kb, between 10 kb to 25 kb, between 10 kb to 20 kb, between 10 kb to 15 kb, between 15 kb to 30 kb, between 15 kb to 25 kb, between 15 kb to 20 kb, between 20 kb to 30 kb, between 20 kb to 25 kb, or between 25 kb to 30 kb in size.

In some embodiments, the polynucleotide has a GC-content of 10% or greater, 20% or greater, 30% or greater, 40% or greater, 50% or greater, 60% or greater, 70% or greater, 80% or greater, or 90% or greater, wherein the GC-content is the percentage of total bases in the polynucleotide that contain a guanine or cytosine. In certain embodiments, the polynucleotide has a GC content of between 10% and 90%, between 20% and 80%, between 30% and 70%, between 40% and 60%, between 40% and 90%, between 50% and 90%, between 60% and 90%, or between 70% and 90%. In certain embodiments, the polynucleotide has a GC content of 50% or greater. In some embodiments, the polynucleotide has a GC content of 60% or greater. In still other embodiments, the polynucleotide has a GC content of 70% or greater. In certain embodiments, the polynucleotide has a GC content of 80% or greater. In some embodiments, the polynucleotide has a GC content of 90% or greater.

In some embodiments, the polynucleotide is produced with 10 or fewer deletion errors, for example with 9 or fewer deletion errors, 8 or fewer deletion errors, 7 or fewer deletion errors, 6 or fewer deletion errors, 5 or fewer deletion errors, 4 or fewer deletion errors, 3 or fewer deletion errors, 2 or fewer deletion errors, 1 or fewer deletion errors, or with no deletion errors, compared to the intended sequence. In certain embodiments, the polynucleotide is produced with no deletion errors. In other embodiments, the polynucleotide is produced with 2 or fewer deletion errors. In certain embodiments, the polynucleotide is produced with 4 or fewer deletion errors. In some embodiments, the polynucleotide is produced with 6 or fewer deletion errors. In still other embodiments, the polynucleotide is produced with 8 or fewer deletion errors.

In some embodiments of the methods and systems described herein, the polypeptide is produced with an accuracy rate of 95% or greater, 96% or greater, 97% or greater, 98% or greater, 99% or greater, 99.5% or greater, 99.9% or greater, 99.95% or greater, or 99.99% or greater, wherein the accuracy rate is the sequence of the produced polynucleotide compared to the intended sequence. In some embodiments, the polypeptide is produced with an accuracy rate of at 98% or greater. In certain embodiments, the polypeptide is produced with an accuracy rate of 99% or greater. In still further embodiments, the polypeptide is produced with an accuracy rate of 99.9% or greater. In some embodiments, the polypeptide is produced with an accuracy rate of 99.99% or greater. In some embodiments, the system produces polypeptides with an average accuracy rate of 95% or greater, 96% or greater, 97% or greater, 98% or greater, 99% or greater, 99.5% or greater, 99.9% or greater, 99.95% or greater, or 99.99% or greater. In some embodiments, the system provided herein produces polypeptides with an average accuracy rate of at 98% or greater. In certain embodiments, the system provided herein produces polypeptides with an average accuracy rate or 99% or greater. In still further embodiments, the system provided herein produces polypeptides with an average accuracy rate of 99.9% or greater. In some embodiments, the system provided herein produces polypeptides with an average accuracy rate of 99.99% or greater.

In some embodiments of the method provided herein, after extending the primer to generate the polynucleotide, the polynucleotide is cleaved from the solid support. For example, in certain embodiments, the polynucleotide is cleaved enzymatically. In some embodiments, the polynucleotide is cleaved by a restriction enzyme, for example a Type IIS restriction enzyme. In other embodiments, the polynucleotide is not cleaved from the solid support. In certain embodiments, the polynucleotide remains attached to the solid support, and is used in one or more subsequent reactions.

In certain embodiments of the method provided herein, after extending the primer to generate a single stranded polynucleotide, the polynucleotide is combined with a reverse primer and the reverse primer is extended to generate a second polynucleotide complementary to the single stranded polynucleotide. Thus, in some embodiments, after extending the primer to generate a single stranded polynucleotide, the polynucleotide is combined with a reverse primer and the reverse primer is extended to generate double stranded polynucleotide.

III. Templates

Further provided herein is a method of selecting templates. Depending on the sequences of the templates and primer, two or more unique templates in the same well may have undesired interactions with each other and/or with the primer. For example, in some embodiments two or more unique templates in the same well may bind to each other, reducing the opportunity for a template to bind to the primer and inhibiting extension of the 3′ growing end. In other embodiments, a template may bind to the primer in an undesired location, which may also inhibit extension of the 3′ end. These undesired interactions may, in some embodiments, decrease efficiency, speed, and/or fidelity of generating the polynucleotide. In some embodiments, selecting two or more unique templates to be combined in one template well includes minimizing unintended interactions. In certain embodiments, it includes maximizing the number of unique templates while minimizing unintended interactions. Combining a greater number of templates in one well may allow for a smaller array size with fewer total template wells, which may increase speed of polynucleotide generation.

Thus, provided herein are methods of selecting templates. In some embodiments, these methods decrease undesired interactions between two or more templates in one well. In some embodiments, the method of selecting templates comprises one or more of: selecting optimal template sequence, identification of templates to be combined in the same well, and tuning of template characteristics.

a. Designing Templates

In some embodiments, the operating temperature for the extension of the primer is selected such that the polymerase exhibits the maximum rate of primer extension and/or the minimum error rate. In some embodiments, this operating temperature depends upon the identity of the polymerase used, the presence of one or more cofactors, and/or the concentration of one or more of salt, template, and growing primer. Once the operating temperature range has been identified, templates with a Tₘ within that range may be selected.

In one embodiment, the template selection method begins by selecting the binding sequences to which an overhang region will be added. This may be done by identifying binding sequences of X bases, where X is the minimum number of bases necessary for the polymerase to function. For all possible binding sequences, each permutation of X bases is considered. In some embodiments, a subset of all permutations of X bases is considered to, for example to develop an array with a subset of all the possible sequences. The predicted Tₘ is calculated for each of these possible X base sequences, taking into account the other contents of the solution (for example, concentration of salt, template, and/or growing primer). These calculations can be repeated to determine the predicted Tₘ of the templates with X+1 bases, wherein the identity of the +1 base may be different for each calculation (for example, the +1 base may be A, T, G, or C, or another base as described herein). Then, for a given operating temperature range, the binding sequences (X or X+1) that fall within the range are identified. If no X or X+1 sequences fall within the range, the calculations can be repeated for X+2, X+3, X+4, etc. sequences until a binding sequence with the closest Tₘ to the optimal operating temperature range is identified for each template.

Once the binding sequence for the template has been identified, the overhang region sequence is added to the binding sequence to produce a template that can be used to extend a primer. To generate a full list of templates which could be used to incorporate any nucleotide, it is necessary to combine the binding sequence with each possible overhang region. For example, to generate the four templates that could be used to incorporate each of the four standard bases separately into a given growing primer, the same binding sequence must be combined with one of four complementary standard nucleotide overhangs. For example, if the binding sequence is “GGGG,” the full templates with binding sequence and overhang would be “GGGGA” (to incorporate T into the primer), “GGGGG” (to incorporate C into the primer), “GGGGT” (to incorporate A into the primer), and “GGGGC” (to incorporate G into the primer). A similar method may be used to generate templates that comprise one or more non-standard bases, or for templates for use in incorporating one or more non-standard bases into the growing primer.

In some embodiments, the 5′ overhang region of the template is not included when calculating the Tₘ of the templates. In other embodiments, the 5′ overhang region of the template is included when calculating the Tₘ of the templates.

In some embodiments, all possible templates are considered in the selection process, for example to develop an array for use in generating many polynucleotides of different sequences. In other embodiments, the selection method is used to identify only a subset of all possible templates, for example to develop and array to generate one polynucleotide sequence, or a plurality of polynucleotides of similar sequence.

In some embodiments, the Tₘ of one or more templates with X bases (minimum number of bases necessary for a particular polymerase to function) is greater than the operating temperature range for that polymerase. In such situations, in some embodiments, the reaction conditions (such as salt concentration) and/or polymerase may be changed to decrease the Tₘ of the templates or to allow for the use of shorter templates. In some embodiments, the presence of one or more cofactors, and/or the concentration of one or more of salt, template, and growing primer may be adjusted to affect the Tₘ of one or more templates without changing the operating temperature. For example, for a given polymerase, some changes to salt concentration may not affect the operating temperature but will affect the Tw of a given template, while a greater or different change to salt concentration could affect the operating temperature.

For example, in certain embodiments, when the Tₘ of one or more templates with X bases is greater than the operating temperature for that particular polymerase, a different polymerase is used. In certain embodiments, a polymerase with a higher operating temperature is used, and the Tₘ for the template falls within this higher operating temperature. In other embodiments, a polymerase that requires fewer bases to function is used, and therefore shorter templates (with lower Tₘ) can be used. In certain embodiments, a polymerase with both a higher operating temperature and which requires fewer bases to function is used, and this combination allows for the selection of a template with a Tₘ that falls within the operating temperature (for example, through a combination of fewer bases and higher operating temperature). In certain embodiments, two or more wells of the array comprise different polymerases, with different operating temperatures, different minimum required bases for binding and extension of the primer, or both. In certain embodiments, the wells of the array comprise two or more, three or more, or four or more different polymerases, with each polymerase in a separate well.

In some embodiments, one or more conditions of the solution in a well is adjusted to change the Tₘ of a template. For example, increasing the pH of the solution may, in some embodiments, increase Tₘ of a given template. Increasing the salt concentration of the solution may, in some embodiments, increase the Tₘ of a given template. Adding or increasing the concentration of one or more divalent cations (such as Mg²⁺) may also increase the Tₘ of a given template in some embodiments, and this increase may be greater than if adding or increasing the concentration of a monovalent cation. Thus, in certain embodiments, if the Tₘ a template with X bases (minimum number of bases necessary for a particular polymerase to function) is greater than the operating temperature range for that polymerase, the pH or salt concentration is decreased to decrease the Tₘ of the template.

In still other embodiments, the concentration of one or more templates in the same solution can also affect Tₘ. Thus, in certain embodiments, if the Tₘ of a given template with X bases (minimum number of bases necessary for a particular polymerase to function) is greater than the operating temperature range for that polymerase, the presence of one or more templates in the same well or changing the concentration of one or more templates in the same well may change the Tₘ of the given template such that it is within the operating temperature of the polymerase. For example, in some embodiments, increasing the concentration of a template increases the effective Tₘ of that template, or increases the percentage of that template that is hybridized at a given temperature.

In some embodiments, the pH, overall salt concentration, divalent cation concentration, concentration of one or more cofactors, and/or concentration of one or more templates is adjusted such that the Tₘ of each template in one well is within 10° C., within 5° C., within 4° C., within 3° C., within 2° C., or within 1° C. of the operating temperature of the polymerase (which may, for example, be the temperature of the solution during the hybridization step, or the temperature of the solution during the extension step, or both). In some embodiments, the pH, overall salt concentration, divalent cation concentration, concentration of one or more cofactors, and/or concentration of one or more templates in the solution of two or more wells of the array is adjusted for different operating temperatures. This may be done, for example, when two or more wells of the array comprise different polymerases in solution.

b. Undesired Interactions

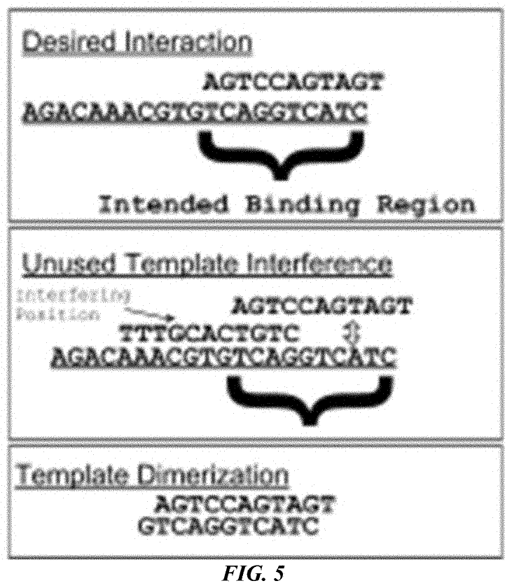

In some embodiments, templates are selected to reduce undesired interactions between the templates themselves. These interactions may include, for example, homodimerization (FIG. 5, bottom panel).

In other embodiments, templates are selected to reduce undesired interactions between one or more templates and the primer. These interactions may include, for example, detrimental interactions between the complementary template and the primer, such as binding of the complementary template to a location on the primer that prevents or inhibits 3′ extension. For example, if a complementary template has a 10 base complementary region, and binding of this complementary region to the 10 base 3′ end of the primer would result in a desired overhang region (for extension of the primer), binding of the complementary region to just a portion of the 3′ end of the primer without producing the one nucleotide overhang region may inhibit or prevent extension. FIG. 2 demonstrates an undesirable template interaction with 15, wherein the template has hybridized with the primer but in a location that does not produce a template overhang. FIG. 5 also demonstrates one embodiment of an undesirable interaction in the middle panel, comparing the location of binding of template at interfering position (middle sequence binding with primer) with the desired location of binding (top sequence, illustrated with arrows).

Undesirable interactions may in some embodiments be determined based on Tₘ calculations for each potential interaction, for example calculating the Tₘ of both templates bound to each other, and the Tₘ of a template and an interfering position on the growing primer.

Extension of the primer may in some embodiments be delayed or inhibited when a non-complementary template demonstrates binding to a portion of the 3′ end of the primer, but still allows for some binding of the complementary template, allowing the polymerase to extend. This may occur, for example, when the binding energy of the non-complementary template with the primer is similar or only moderately higher than that for the complementary template with the primer. In some embodiments, the extension step can be prolonged to increase the chances that the primer is extended by the complementary template.

In some embodiments, if the binding energy of the non-complementary template with the 3′ end of the primer is more favorable than the binding energy of the complementary template and the 3′ end of the primer, the non-complementary template binding may prevent the complementary template from binding.

In some embodiments, a path-finding algorithm may be used to determine the groupings of templates which minimize undesired interactions. In some embodiments the objective function for this optimization considers the probability and severity of an interference event to (a) minimize sequences that reach a prohibition of synthesis point, and cannot be extended, and (b) reduce the elapsed time added to the synthesis process by non-complementary template-primer binding interfering with complementary template-primer binding and extension.

In some embodiments, templates may be grouped in order to maximize the average percentage of the desired template (such as the complementary template) bound to the growing strand. The percentage of desired template bound may be found using nearest neighbors models to calculate the free energies of the binding of each template to their corresponding growing primer. The free energies of different types of binding of each template to their corresponding growing primer can be calculated—for example, binding of a template to produce the desired overhang region, and binding of the same template in a way that does not produce the desired overhang region or any overhang region. In some embodiments, for each possible grouping of templates, a percentage of desired template is found by calculating the Boltzmann weight from the free energy of the desired template bound with the desired overhang, as well as the Boltzmann weights calculated from the free energies of the undesired templates bound at positions that block the binding site for the desired template. In certain embodiments, the calculation assumes that the region 5′ to the desired template binding site has a sequence which is complimentary to the undesired sequence.

The primer-template binding energy for a given template in given binding conditions (e.g., temperature, primer and template concentration, salt concentration) depends on the identity of the primer. Thus, for example, for a first 3′ primer sequence, a given template may have a relatively higher binding energy, while for a second 3′ primer sequence the same template may have a relatively lower binding energy in the same conditions. In some embodiments, if the sequences of the two or more unique templates in one well are known, the 3′ sequences of primers that would contact that well for hybridization and extension are therefore also known (as they are complementary to the templates), and the binding energies of the possible template-primer pairings can be calculated.

In some embodiments, for the two or more unique templates within one well of the array, the binding energy of the primer and complementary template is independently at least 5% greater, at least 10% greater, at least 15% greater, at least 20% greater, at least 25% greater, at least 30% greater, at least 35% greater, at least 40% greater, at least 45% greater, at least 50% greater, at least 55% greater, at least 60% greater, at least 65% greater, at least 70% greater, at least 75% greater, at least 80% greater, at least 85% greater, at least 901% greater, at least 95% greater, at least 100% greater, or more than 100% greater than the binding energy of the primer and one or more non-complementary templates in the same well.

In some embodiments, for a complementary template and one or more non-complementary templates in the same well, the one or more non-complementary templates independently have a binding energy with the primer that differs by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, or more than 100% compared to the binding energy of the complementary template and the primer. Binding energy may be calculated, for example, using nearest neighbor methods.

c. Tuning of Template Binding

In some embodiments, tuning the binding of templates can be helpful to reduce undesired interactions. For example, if the complementary template is A, the non-complementary template is B, and the growing primer is C, the A and B templates may be selected with a particular Tₘ such that the concentration of the AC and BC binding complexes are as similar as possible at the operating conditions.

In some embodiments, changing concentration of individual templates can be helpful to reduce undesired interactions. For example, if one template has a lower Tₘ than is desirable, increasing the concentration of said template will increase the number of molecules of that template bound to primer at any given temperature. In this way, templates with a lower Tₘ can be more evenly competitive for binding to primer compared with higher melting temperature templates in certain embodiments.

In some embodiments, non-standard bases may be included in a template sequence to increase binding affinity, and therefore increase Tₘ to be within the desired range. For example, in some embodiments the standard bases guanine and tyrosine may be replaced with 8-aza-7-deazaguanosine (Super G) and 5-hydroxybutynl-2′-deoxyuridine (Super T®), respectively, to increase Tₘ of a template.

### EXAMPLES

The following Examples are merely illustrative and are not meant to limit any aspects of the present disclosure in any way.

Templates listed in Table 1 were used in the following examples (obtained from Integrated DNA Technologies (IDT)).

TABLE 1
Template Sequences

| Name | Sequence | Notes |
|---|---|---|
| Oligo | TTGAGCGTGAACTCG | Blocked at 3′ end |
| 1 | | with a C3 Spacer |
| Oligo | ATGAGCGTGAACTCG | Blocked at 3′ end |
| 2 | | with a C3 Spacer |
| Oligo | CTGAGCGTGAACTCG | Blocked at 3′ end |
| 3 | | with a C3 Spacer |
| Oligo | GTGAGCGTGAACTCG | Blocked at 3′ end |
| 4 | | with a C3 Spacer |
| Oligo | ATCACGCGAGTTCACGCTCA | |
| 5 | | |
| Oligo | ATCACGCGAGTTCACGCTCAC | |
| 6 | | |
| Oligo | GTG AGC GTG ANN NN | Blocked at 3′ end |
| 7 | | with a C3 Spacer |
| Oligo | GTG AGC GTN NNN | Blocked at 3′ end |
| 8 | | with a C3 Spacer |
| Oligo | GTG AGC NNN N | Blocked at 3′ end |
| 9 | | with a C3 Spacer |
| Oligo | ATCACGCGAGTTCACGCTCA | Biotin modifica- |
| 10 | | tion on 5′ end |
| Oligo | ATCACGCGAGTTCACGCTCAC | Biotin modifica- |
| 11 | | tion on 5′ end |
| Oligo | ATCACGCGAGTTCACGCTCACC | Biotin modifica- |
| 12 | | tion on 5′ end |
| Oligo | GGTGAGCGTGAACTC | Blocked at 3′ |
| 13 | | end with a C3 Spacer |

### Example 1: Addition of Four Bases to a Growing Primer

This example demonstrates the addition of four bases to a growing primer.

Four sample tubes were prepared according to Table 2 below, and placed on ice. Each tube was then thermocycled using an MJ Research PTC-100® thermocycler according to the following protocol: (a) incubation at 25° C. for 20 minutes, then (b) heat shock at 95° C. for 20 minutes.

TABLE 2
Contents of sample tubes for Example 1

| | Tube # | | | |
|---|---|---|---|---|
| | 1 | 2 | 3 | 4 |
| | Add A | Add G | Add T | Add C |
| Final Volume | 15 | 15 | 15 | 15 |
| Oligo 1 100 uM | 4.5 | 0 | 0 | 0 |
| Oligo 2 100 uM | 0 | 4.5 | 0 | 0 |
| Oligo 3 100 uM | 0 | 0 | 4.5 | 0 |
| Oligo 4 100 uM | 0 | 0 | 0 | 4.5 |
| Oligo 5 600 ng/uL | 0.6 | 0.6 | 0.6 | 0.6 |
| dATP 100 mM | 0.5 | 0 | 0 | 0 |
| dGTP 100 mM | 0 | 0.5 | 0 | 0 |
| dTTP 100 mM | 0 | 0 | 0.5 | 0 |
| dCTP 100 mM | 0 | 0 | 0 | 0.5 |
| 10× klenow buffer | 1.5 | 1.5 | 1.5 | 1.5 |
| Klenow (exo-) | 0.6 | 0.6 | 0.6 | 0.6 |
| water | 7.3 | 7.3 | 7.3 | 7.3 |

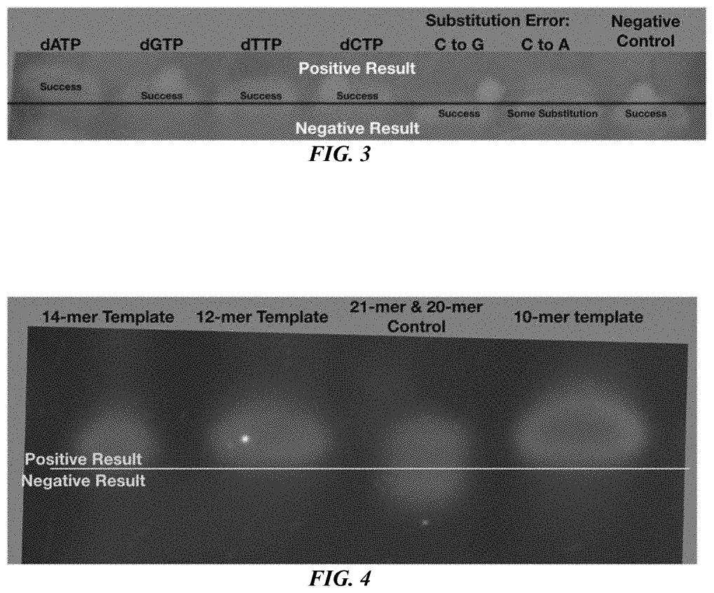

Each sample was then mixed with an equal volume (15 uL) of gel loading dye (95% formamide, 18 mM EDTA, 0.025% SDS, xylene cyanol, and bromophenol blue). The mixed samples were loaded into separate wells of a 15% TBE-Urea polyacrylamide gel (Bio-Rad). In addition, samples of oligo 5 and oligo 6 were also loaded into the gel to serve as a ladder. The gel was subjected to electrophoresis, then removed from the casing and stained in SYBR™ Green II dye solution for 30 minutes. The gel was imaged using a blue light transilluminator. An image of the gel is depicted in FIG. 3.

### Example 2: Addition of a Base to a Growing Primer in the Presence of a Template Mixture

This example demonstrates the addition of a base to a growing primer in the presence of a mixture of templates.

Three sample tubes were prepared according to Table 3 below, and placed on ice. Each tube was then thermocycled using an MJ Research PTC-100® thermocycler according to the following protocol: (a) incubation at 25° C. for 20 minutes, then (b) heat shock at 95° C. for 20 minutes.

TABLE 3
Contents of sample tubes for Example 2

| | Tube # | | |
|---|---|---|---|
| | 1 | 2 | 3 |
| Name | 10-4N | 6-4N | 4-4N |
| Final Volume | 5 | 5 | 5 |
| Oligo 7 4 mM | 3.9 | 0 | 0 |
| Oligo 8 4 mM | 0 | 3.9 | 0 |
| Oligo 9 4 mM | 0 | 0 | 3.9 |
| Oligo 5, 600 ng/uL | 0.3 | 0.3 | 0.3 |
| dCTP 100 mM | 0.1 | 0.1 | 0.1 |
| 10× klenow buffer | 0.5 | 0.5 | 0.5 |
| klenow | 0.2 | 0.2 | 0.2 |

Each sample was then mixed with an equal volume (15 uL) of gel loading dye (95% formamide, 18 mM EDTA, 0.025% SDS, xylene cyanol, and bromophenol blue). The mixed samples were loaded into separate wells of a 15% TBE-Urea polyacrylamide gel (Bio-Rad). In addition, samples of oligo 5 and oligo 6 were also loaded into the gel to serve as a ladder. The gel was subjected to electrophoresis, then removed from the casing and stained in SYBR™ Green 11 dye solution for 30 minutes. The gel was imaged using a blue light transilluminator. An image of the gel is depicted in FIG. 4.

### Example 3: Addition of Multiple Bases in Succession, in the Presence of a Template

This example demonstrates the addition of multiple bases in succession to a growing primer, in the presence of a template.

Following the manufacturer's protocol, single stranded primer was bound to DYNABEADS® M-280 Streptavidin magnetic beads (ThermoFisher). First, the beads were resuspended beads in the vial by vortexing for >30 seconds. Then, 25 uL of 5 mg/mL beads was transferred to a 1.5 mL tube. An equal volume or at least 1 mL of 1× washing buffer was added and the mixture vortexed for 5 sec. The beads were collected by a magnet for 1 minute and the supernatant discarded. The beads were resuspended again in 50 ul of 2× washing buffer, and vortexed briefly. The desired primer (Oligo 10) was prepared at a volume of 50 uL and concentration of 1 uM. The primer (Oligo 10) was combined with the beads, and incubated for 10 minutes using gentle rotation.

Then, the beads were separated using a magnet for 2-3 minutes. A wash process was performed twice in 1× B&W buffer by: (i) Adding 1 mL of 1× washing buffer, vortexing for 5 seconds; and then (ii) Placing the tube on a magnet for 1 minute and discarding supernatant. Finally, the beads were resuspended to the desired concentration, at a volume of 10-uL for Oligo 10.

To carry out the base addition reactions, sample tubes were prepared on ice, according to Table 4 below.

TABLE 4
Contents of sample tubes for Example 3

| | Volume in uL | |
|---|---|---|
| Run Column Number | Sample 1 | Sample 2 |
| Final Vol (uL) | 15 | 14 |
| Oligo 11 on beads | 10 | |
| Oligo 4 at 100 uM | 2.5 | |
| Oligo 13 at 100 nM | | 2.5 |
| dCTP 100 mM | 0.5 | 0.5 |
| 10× klenow buffer | 1.5 | 1.5 |
| Klenow (exo-) | 0.5 | 0.5 |
| Distilled water | | 9 |

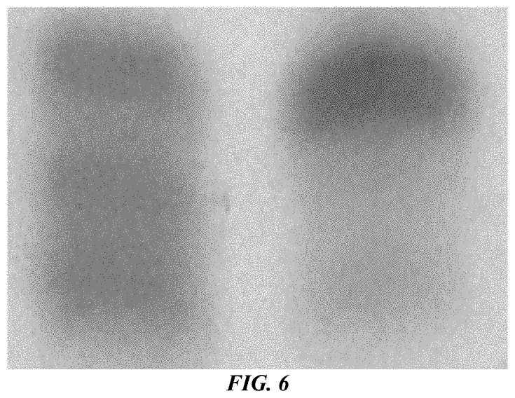

While sample 2 was kept chilled, sample 1 was thermocycled on an MJ Research PTC-100® thermocycler using the following protocol: (a) incubation at 20° C. for 20 minutes, then (b) heat shock at 95° C. for 10 minutes. Sample 1 was removed from the heating block, placed on a magnet for 1 minute, the supernatant removed, and then sample 2 was added to sample 1. The combined sample was then thermocycled again following the same protocol. The combined sample was removed from the heating block, placed on a magnetic rack for 1 minute, supernatant removed, and 15 uL loading dye added (95% formamide, 18 mM EDTA, 0.025% SDS, xylene cyanol, and Bromophenol Blue). The sample was loaded into a 15% TBE-Urea gel (Bio-Rad), and a ladder sample of oligos 10, 11, and 12 was added to a separate well. Following electrophoresis, the gel was removed from the casing and stained in SYBR™ Green 11 dye solution for 30 minutes, then imaged on a blue light transilluminator. The gel image is shown in FIG. 6.

### Example 4: Addition of Successive Bases in the Presence of a Template on an Automated Device

This example demonstrates the addition of successive bases to a growing primer using an automated device. Following the manufacturer's protocol, single stranded primer (Oligo 10) will be bound to DYNABEADS® M-280 Streptavidin magnetic beads (ThermoFisher), as described in Example 3 above. One or more beads with primer attached are attached to a magnetized needle, which is controlled by a motor. On ice, samples are prepared according to Table 5 below.

TABLE 5
Contents of samples used in Example 4

| | Volume in uL | |
|---|---|---|
| Run Column Number | Sample 1 | Sample 2 |
| Final Vol (uL) | 15 | 15 |
| Oligo 4 at 100 uM | 2.5 | |
| Oligo 13 at 100 uM | | 2.5 |
| dCTP 100 mM | 0.5 | 0.5 |
| 10× klenow buffer | 1.5 | 1.5 |
| Klenow (exo-) | 0.5 | 0.5 |
| Distilled water | 10 | 10 |

Sample 1 and sample 2 are placed in separate wells of an array. The needle with the magnetic bead(s) is lowered into the sample 1 well, and the well heated to 20° C. for 20 minutes. The needle with bead(s) is then removed from the sample 1 well, and washed by dipping into distilled water for 2 minutes at 25° C. The needle with bead(s) is lowered into the sample 2 well, the well heated to 20° C. for 20 minutes, then the needle with bead(s) removed and washed by dipping into distilled water for 2 minutes at 25° C. Finally, the needle with bead(s) is lowered into a well containing 15 uL of loading dye (95% formamide, 18 mM EDTA, 0.025% SDS, xylene cyanol, and Bromophenol Blue). The contents of the loading dye well is loaded into a 15% TBE-Urea gel (Bio-Rad), and a ladder sample (Oligos 10, 11, and 12) is loaded into a separate well. The gel undergoes electrophoresis, then is removed from its casing and stained in SYBR™ Green II dye solution for 30 minutes, followed by imaging on a blue light transilluminator.

What is claimed is:

1. A method of extending a primer to generate a polynucleotide, comprising:
(a) providing a primer, wherein the primer has a 5′ fixed end and a 3′ growing end, wherein the fixed end is bound to a solid substrate;
(b) providing an array comprising a plurality of template wells, wherein at least a portion of the wells each comprise two or more unique templates in solution;
(c) contacting the solid substrate with the solution of a well comprising two or more unique templates,
wherein one of the two or more unique templates is a complementary template comprising a complementary region, wherein the sequence of the complementary region is complementary to the growing end; and
at least one of the two or more unique templates is a non-complementary template, wherein the sequence of the non-complementary template is not complementary to the growing end;
(d) hybridizing the growing end and the complementary region to provide an overhang of the complementary template comprising one or more nucleotides;
(e) extending the growing end of the primer by the addition of one or more nucleotides, wherein the one or more added nucleotides are complementary to the one or more nucleotides of the overhang region;
(f) dehybridizing the growing end and the complementary region; and
(g) repeating steps (c) through (f) to generate the polynucleotide.

2. The method of claim 1, wherein the one or more nucleotides are added by a template-dependent DNA polymerase.

3. The method of claim 1 or 2, further comprising washing the solid substrate after step (f).

4. The method of any one of claims 1 to 3, further comprising heating the solution in step (d) to hybridize the growing end and the complementary region.

5. The method of claim 4, wherein the solution is heated to within 20° C. of the Tₘ of the complementary template.

6. The method of any one of claims 1 to 5, further comprising cooling the solution in step (f) to dehybridize the growing end and the complementary region.

7. The method of any one of claims 1 to 6, wherein the polynucleotide is at least 300 bases in size.

8. The method of any one of claims 1 to 7, wherein the polynucleotide comprises a GC content of 80% or greater.

9. The method of any one of claims 1 to 8, wherein the polynucleotide comprises fewer than 6 deletion errors.

10. The method of any one of claims 1 to 9, wherein the overhang region comprises one nucleotide.

11. The method of any one of claims 1 to 10, wherein the polypeptide is produced with an accuracy rate of 98% or greater.

12. The method of any one of claims 1 to 11, wherein the two or more unique templates independently comprise between 6 bases to 12 bases.

13. The method of any one of claims 1 to 12, wherein the one or more non-complementary templates independently have a binding energy that differs by at least 5% compared to the binding energy of the complementary template.

14. A system for extending a growing primer to generate a polynucleotide, comprising:
a growing primer comprising a 5′ fixed end and a 3′ growing end, wherein the fixed end is bound to a solid substrate;
a template array comprising a plurality of template wells, wherein at least a portion of the template wells each comprise two or more unique templates in solution,
wherein in one of the template wells, one of the two or more unique templates is a complementary template comprising a complementary region, wherein the sequence of the complementary region is complementary to the growing end; and
at least one of the two or more unique templates is a non-complementary template, wherein the sequence of the non-complementary template is not complementary to the growing end; and
a positioning apparatus configured to contact the solid substrate with the solution of a well.

15. The system of claim 14, comprising no more than one million template wells each comprising two or more unique templates in solution.

16. The system of claim 14 or 15, further comprising a washing well.

17. The system of any one of claims 14 to 16, wherein the positioning apparatus further comprises a heating element.

18. The system of any one of claims 14 to 17, wherein the template array further comprises one or more heating elements.

19. The system of any one of claims 14 to 18, wherein the two or more unique templates independently comprise between 6 bases to 12 bases.

20. The system of any one of claims 14 to 19, wherein the one or more non-complementary templates has a binding energy that differs by at least 5% compared to the binding energy of the complementary template.
